(19) United States
(12) Patent Application Publication
(10) Pub. No.: US 2008/0071269 A1
(43) Pub. Date: Mar. 20, 2008

(54) Curved Endoscopic Medical Device

(75) Inventors: Estela H. Hilario, Los Altos, CA (US); Russel M. Sampson, Palo Alto, CA (US); Robert Kotmel, Burlingame, CA (US)

Correspondence Address: CYTYC CORPORATION, 250 CAMPUS DRIVE, MARLBOROUGH, MA, 01752, US

(73) Assignee: Cytyc Corporation, Marlborough, MA (US)

(21) Appl. No.: 11/532,886

(22) Filed: Sep. 18, 2006

(51) Int. Cl. A61B 18/18

(52) U.S. Cl. 606/50

(57) ABSTRACT

A medical device and procedure is described which can be used for occluding a fallopian tube. In one implementation, the apparatus includes an elongate member, an electrode carrier and one or more conductors. The elongate member has a lumen operable to couple to a vacuum source and draw moisture way from one or more electrodes included in the electrode carrier, and a lumen configured to receive a hysteroscope. The electrode carrier includes one or more bipolar electrodes and can to couple to a radio frequency energy generator. The one or more conductors connect to a controller operable to control the delivery of radio frequency energy to the one or more bipolar electrodes. The elongate member is a substantially rigid member configured with a curve to facilitate advancement of the distal end transcervically through a uterus and into a region of a tubal ostium of a fallopian tube to be occluded.

### TECHNICAL FIELD

This invention relates to a medical device and procedure.

### BACKGROUND

Medical procedures occurring within the body often require the aid of visualization either before, during and/or after the procedure. For example, procedures including localized medicant delivery, energy delivery, biopsy and the like. One medical procedure that can benefit from direct visualization is in situ tissue ablation through the application of radio frequency energy. An endoscope is one such device used for visualization, and conventionally includes a straight, rigid shaft that can be inserted into a patient either through a natural orifice or an incision.

### SUMMARY

This invention relates to a medical device and procedure. In general, in one aspect, the invention features an apparatus for occluding a fallopian tube. The apparatus includes an elongate member, an electrode carrier and one or more conductors. The elongate member has a distal end, a proximal end and a central interior including at least a first lumen operable to couple to a vacuum source and to draw moisture way from one or more electrodes included in the electrode carrier positioned at the distal end of the elongate member and at least a second lumen configured to receive a hysteroscope. The first lumen and the second lumen can be the same lumen or can be separate lumens. The electrode carrier attaches to the distal end of the elongate member and includes one or more bipolar electrodes formed thereon and is operable to couple to a radio frequency energy generator. The one or more conductors extend from the electrode carrier to the proximal end of the elongate member and are configured to connect to a controller operable to control the delivery of radio frequency energy to the one or more bipolar electrodes. The elongate member is a substantially rigid member configured with a curve to facilitate advancement of the distal end transcervically through a uterus and into a region of a tubal ostium of a fallopian tube to be occluded.

Implementations of the invention can include one or more of the following features. The apparatus can include a hysteroscope positioned within the first lumen of the elongate member, such that a distal end of the hysteroscope is positioned approximately just proud of a distal end of the electrode carrier. The hysteroscope can be substantially rigid and configured with a similar curve to the curve of the elongate member. Alternatively, the hysteroscope can be substantially flexible and can flex to accommodate the curve of the elongate member. The electrode carrier can include an approximately cylindrically shaped support member within a fabric sheath having conductive metallized regions and one or more non-conductive regions formed thereon to create the one or more bipolar electrodes. The support member can be formed from a plastic material, the fabric sheath can be formed from a polymer mesh and the conductive metallized regions can be formed by selectively coating the polymer mesh with gold. The polymer forming the polymer mesh can be a combination of nylon and spandex.

The electrode carrier can be an approximately cylindrically shaped member including a metallic mesh insert molded in a support member formed from a plastic material, where the metallic mesh forms conductive regions and the plastic material forms non-conductive regions thereby creating the one or more bipolar electrodes. The metallic mesh insert can be formed from a stainless steel material or a platinum material. The electrode carrier can include an approximately cylindrically shaped support member having a diameter in the range of approximately five to 10 millimeters.

The apparatus can further include a vacuum source in fluid communication with the first lumen included in the elongate member and operable to draw tissue surrounding the electrode carrier into contact with the one or more bipolar electrodes and to draw moisture generated during delivery of the radio frequency energy to the one or more bipolar electrodes away from the one or more bipolar electrodes and to substantially eliminate liquid surrounding the one or more bipolar electrodes.

The apparatus can further include a radio frequency energy generator coupled to the one or more bipolar electrodes through the one or more conductors, where the radio frequency energy generator includes or is coupled to a controller operable to control the delivery of radio frequency energy to the one or more bipolar electrodes.

In general, in another aspect, the invention features an apparatus for occluding a fallopian tube including a hysteroscope, an elongate member, an electrode carrier and one or more conductors. The hysteroscope includes a working channel extending from a distal end to a proximal end, where the hysteroscope is substantially rigid and configured with a curve to facilitate advancement of the distal end transcervically through a uterine cavity and into a region of a tubal ostium of a fallopian tube to be occluded. The elongate member is positioned within the working channel of the hysteroscope, and has a distal end, a proximal end and a central interior. The central interior includes a lumen operable to couple to a vacuum source and to draw moisture way from one or more electrodes included in an electrode carrier positioned at the distal end of the elongate member. The elongate member is a substantially rigid member configured with a curve similar to the curve of the hysteroscope to facilitate advancement of the distal end of the elongate member to the distal end of the hysteroscope. The electrode carrier is attached to the distal end of the elongate member and includes one or more bipolar electrodes formed thereon and operable to couple to a radio frequency energy generator. The one or more conductors extend from the electrode carrier to the proximal end of the elongate member and are configured to connect to a controller operable to control the delivery of radio frequency energy to the one or more bipolar electrodes.

In general, in another aspect, the invention features an apparatus for ablating tissue including an elongate member, an electrode carrier and one or more conductors. The elongate member has a distal end, a proximal end and a central interior including at least a first lumen operable to couple to a vacuum source and to draw moisture way from one or more electrodes included in an electrode carrier positioned at the distal end of the elongate member and at least a second lumen configured to receive an endoscope. The electrode carrier is attached to the distal end of the elongate member and includes one or more bipolar electrodes formed thereon and operable to couple to a radio frequency energy generator. The one or more conductors extend from the electrode carrier to the proximal end of the elongate member and are configured to connect to a controller operable to control the delivery of radio frequency energy to the one or more bipolar electrodes. The elongate member is a substantially rigid member configured with a curve to facilitate advancement of the distal end through a body cavity to a region of tissue to be ablated.

In general, in another aspect, the invention features an apparatus for ablating tissue including an endoscope, an elongate member, an electrode carrier and one or more conductors. The endoscope includes a working channel extending from a distal end to a proximal end. The endoscope is substantially rigid and configured with a curve to facilitate advancement of the distal end through a body cavity to a region of tissue to be ablated. The elongate member is positioned within the working channel of the endoscope and has a distal end, a proximal end and a central interior including a lumen operable to couple to a vacuum source and to draw moisture way from one or more electrodes included in an electrode carrier positioned at the distal end of the elongate member. The elongate member is a substantially rigid member configured with a curve similar to the curve of the hysteroscope to facilitate advancement of the distal end of the elongate member to the distal end of the endoscope. The electrode carrier is attached to the distal end of the elongate member and includes one or more bipolar electrodes formed thereon and operable to couple to a radio frequency energy generator. The one or more conductors extend from the electrode carrier to the proximal end of the elongate member and are configured to connect to a controller operable to control the delivery of radio frequency energy to the one or more bipolar electrodes.

In general, in another aspect, the invention features an apparatus for occluding a fallopian tube including an elongate member, an electrode carrier and one or more conductors. The elongate member has a distal end, a proximal end and a central interior including at least a first lumen operable to couple to a vacuum source and to draw moisture way from one or more electrodes included in an electrode carrier positioned at the distal end of the elongate member and at least a second lumen configured to receive a hysteroscope. The first lumen and the second lumen can be the same lumen or can be separate lumens. The electrode carrier is attached to the distal end of the elongate member and includes one or more bipolar electrodes formed thereon and operable to couple to a radio frequency energy generator. The electrode carrier has a substantially cylindrical shape. The one or more conductors extend from the electrode carrier to the proximal end of the elongate member and are configured to connect to a controller operable to control the delivery of radio frequency energy to the one or more bipolar electrodes. The elongate member includes an aperture formed in a sidewall of the elongate member toward a distal end of the elongate member but proximate to the electrode carrier. The aperture is configured to allow a distal end of the hysteroscope to pass through, providing the hysteroscope with a field of view extending from a side of the elongate member.

In one implementation, the elongate member is flexible and receiving the hysteroscope in the second lumen causes the elongate member to bend off axis forming a curvature in the elongate member.

In general, in another aspect, the invention features an apparatus for occluding a fallopian tube including an elongate member, an electrode carrier and one or more conductors. The elongate member has a distal end, a proximal end and a central interior including at least a first lumen operable to couple to a vacuum source and to draw moisture way from one or more electrodes included in an electrode carrier positioned at the distal end of the elongate member and at least a second lumen configured to receive a rigid and curved hysteroscope. The first lumen and the second lumen can be the same lumen or can be separate lumens. The electrode carrier is attached to the distal end of the elongate member and includes one or more bipolar electrodes formed thereon and operable to couple to a radio frequency energy generator. The one or more conductors extend from the electrode carrier to the proximal end of the elongate member and are configured to connect to a controller operable to control the delivery of radio frequency energy to the one or more bipolar electrodes. The elongate member is a substantially flexible member configured to bend into a curved configuration upon receiving the rigid and curved hysteroscope in the second lumen, where the curve facilitates advancement of the distal end transcervically through a uterus and into a region of a tubal ostium of a fallopian tube to be occluded.

In general, in another aspect, the invention features a method for fallopian tubal occlusion. A substantially rigid, curved elongate member including a substantially cylindrically shaped electrode carrier positioned at a distal end with one or more bipolar electrodes formed thereon is inserted into a uterine cavity. The electrode carrier is positioned at a tubal ostium of a fallopian tube, such that a distal end of the electrode carrier advances into the tubal ostium. Radio frequency energy is passed through the one or more bipolar electrodes to the tubal ostium to destroy tissue to a known depth and to precipitate a healing response in surrounding tissue that over time scars and occludes the fallopian tube. Implementations of the invention can include one or more of the following features. Passing radio frequency energy through the one or more bipolar electrodes can include passing a current at an initial current level through the one or more bipolar electrodes to the target tissue site to apply an initial power density to destroy tissue for an initial time period and, after the initial time period, ramping up the power density by increasing the current passed through the one or more bipolar electrodes to the target tissue site for a second time period. Ramping up the power density can include gradually increasing the current over the second time period or suddenly increasing the current from the initial current level to a second current level and applying the second current level for the second time period. An impedance level at an interface between the electrode carrier and the tubal ostium can be monitored, where the initial time period is a time period after which a threshold decrease in the impedance level from an initial impedance level is detected. Alternatively, the initial time period can be determined empirically as a time period after which an initial depth of tissue destruction has been achieved

Implementations of the invention can realize one or more of the following advantages. The curvature of the endoscopic medical device allows for easier navigation to a target tissue site. In the implementation of an ablation device including a lumen to receive a curved hysteroscope or a semi-flexible or flexible hysteroscope, where the curvature facilitates positioning the device at a tubal ostium and the position of the optics within the device facilitate device alignment by the operator. Precise positioning of the device can provide improved ablation results and can avoid uterine perforations.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1A shows an ablation device.

FIG. 1B shows the ablation device of FIG. 1A positioned in a uterus.

FIG. 1C is a schematic representation of a region of ablated tissue in a uterus and tubal ostium.

FIG. 2 is a schematic block diagram of a system for tubal occlusion.

FIG. 3A shows the ablation device of FIG. 1A connected to a coupling assembly.

FIG. 3B is a cutaway view of a portion of the ablation device shown in FIGS. 1A and 3A.

FIG. 3C is a cross-sectional view of an RF applicator head of the ablation device shown in FIGS. 1A and 3A.

FIG. 3D is a cross-sectional view of the ablation device shown in FIG. 1A.

FIG. 3E shows an exploded view of a sheath and a distal component of the ablation device shown in FIG. 1A.

FIG. 4A shows an RF applicator head.

FIG. 4B shows a schematic representation of an electrode carrier.

FIG. 5 shows an alternative RF applicator head.

FIG. 6 is a flowchart showing a process for tubal occlusion.

FIG. 7 shows an alternative embodiment of an ablation device.

Like reference symbols in the various drawings indicate like elements.

### DETAILED DESCRIPTION

A method and a system are described that provide a curved endoscopic medical device. Certain areas of the human body that require visualization before or during the performance of a medical procedure can be difficult to access using a conventional straight and rigid endoscope. Flexible endoscopes generally make use of fiber optics, with a narrower field of view than a conventional endoscope and poorer quality resolution. A curved endoscopic medical device is provided that includes both endoscope functionality as well as functionality to perform a medical procedure. The medical device is rigidly formed with a curve to facilitate access to certain areas of the human body. In one implementation, the curved endoscopic medical device includes a rigid, curved endoscope with a working channel configured to house a tool for performing a medical procedure. In another implementation, a curved, rigid tool for performing a medical procedure includes a working channel configured to receive an endoscope, where the endoscope is either rigid and curved similarly to the tool, or is a flexible and can adapt to the curve of the tool.

In one implementation, the medical procedure to be performed by the tool is tissue ablation. In a particular implementation, the tissue ablation is adapted for the purpose of occluding a female's tubal ostium leading from the uterine cavity to the fallopian tubes, thereby sterilizing the female. For illustrative purposes the curved endoscopic device shall be described in the context of an embodiment that can be configured for use within a uterine cavity to occlude one or more fallopian tubes. However, it should be noted that other implementations are possible, and that the curved endoscopic device is not limited to the particular application described. For example, the curved endoscopic device can be used in the area of the nasal passages to remove polyps. In an alternative application, the curved endoscopic device can be used in the area of the trachea during an intubation procedure. For example, a flexible endotracheal tube can be placed over a curved rigid endoscope to facilitate an intubation procedure.

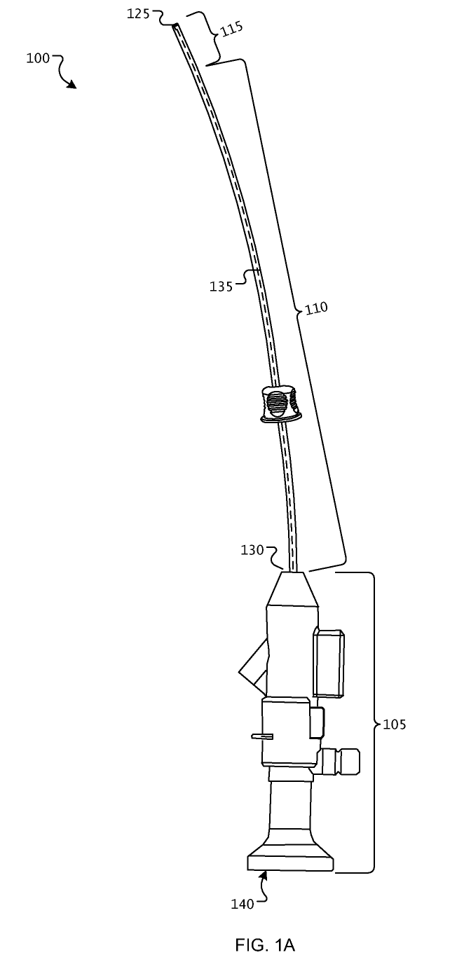

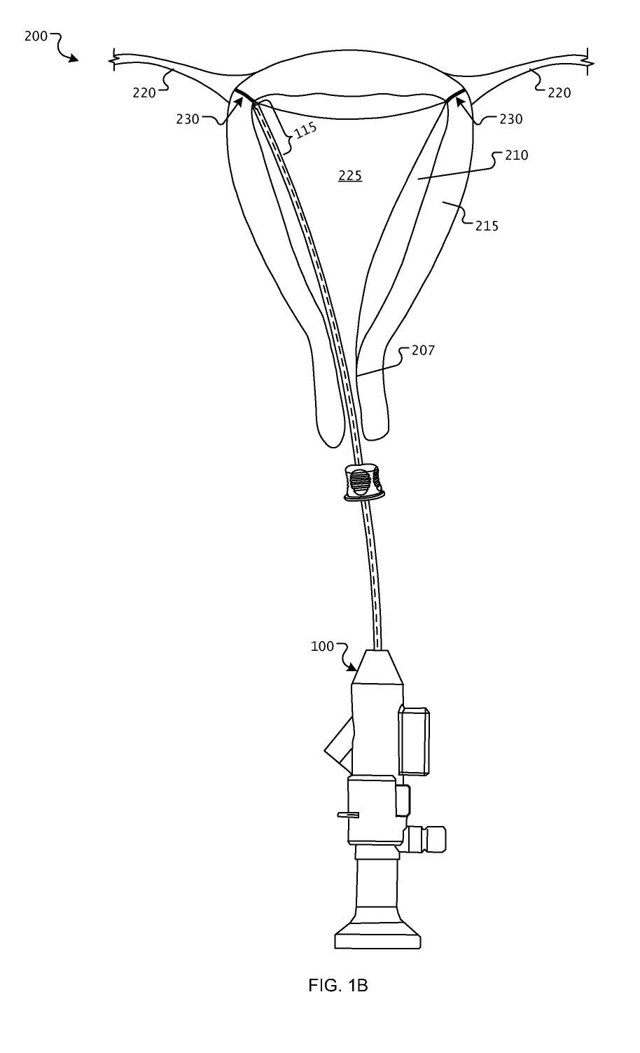

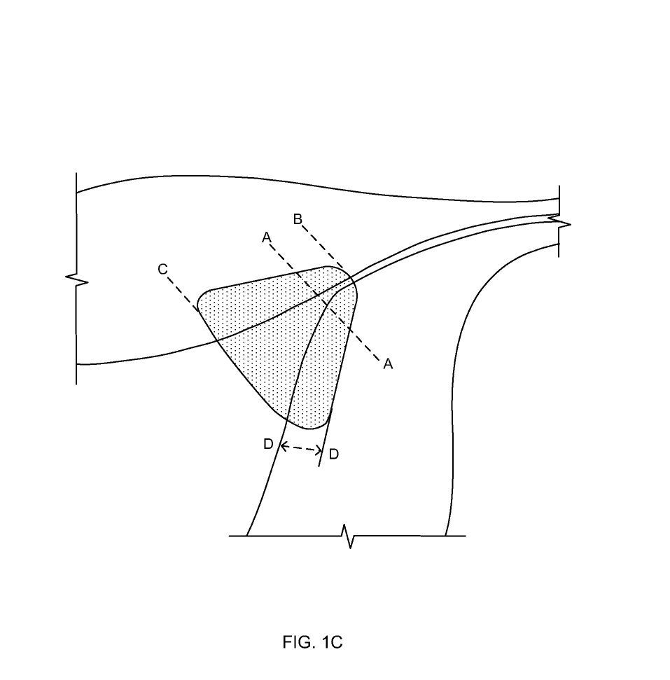

Referring to FIG. 1A, a schematic representation of an ablation device 100 is shown. The ablation device 100 generally includes three major components: a handle 105, a curved shaft 110, and a radio frequency (RF) applicator head 115. The curved shaft 110 includes a distal end 125, a proximal end 130, and a hollow central interior 135. The curved shaft 110 is a substantially rigid member configured with a curve to facilitate the advancement of the distal end 125 through a body cavity to a region of tissue to be ablated. The central interior 135 of the curved shaft 110 includes one or more lumens. For example, the central interior 135 can include a lumen that can be operated so as to couple a vacuum source to the RF applicator head 115 positioned at the distal end 125 of the elongate member 120. The vacuum can be used to draw moisture away from one or more electrodes that can comprise at least a portion of the RF applicator head 115. Additionally, a lumen (either the same lumen that couples to a vacuum source or a different lumen) can be configured to receive a curved hysteroscope. In the particular implementation shown, the ablation device 100 is configured to facilitate entry into a uterine cavity to perform a tubal occlusion procedure and the curved endoscope is a hysteroscope.

The RF applicator head 115 is positioned at the distal end 125 of the curved shaft 110 and includes an electrode carrier having one or more bipolar electrodes. One or more electrical conductors extend from the RF applicator head 115 to the proximal end 130 of the curved shaft 110 and electrically couple the RF applicator head 115 to a controller. The controller can be operated so as to control the delivery of RF energy to the one or more bipolar electrodes.

Referring to FIG. 1B, a schematic representation of a uterus 200 is shown with the ablation device 100 positioned within the uterus 200. The uterus includes a uterine cavity 225, and an internal os 207 both surrounded by uterine tissue, namely endometrial tissue 210 and myometrial tissue 215. The fallopian tubes 220 connect to the uterine cavity 225 at the tubal ostia 230. The ablation device 100 is configured for use within a uterine cavity 225 to occlude one or more of the tubal ostia 230. Occluding the tubal ostia 230 prevents sperm from entering the fallopian tubes 220 and fertilizing an egg, thereby sterilizing the female.

The RF applicator head 115 is introduced transcervically into the uterine cavity and positioned at a tubal ostium 230. Transmitting RF energy through the RF applicator head 115 ablates the uterine tissue 210, 215 and the tissue within the tubal ostium 230. Following the destruction of the tissue at the tubal ostium 230, the healing response occludes the tubal ostium 230 and the adjacent portion of the fallopian tube 220 resulting in sterilization. Referring to FIG. 1C, the targeted tissue destruction from A-A to B is approximately 1.5 to 2.5 millimeters, from A-A to C is approximately 10 to 20 millimeters and the depth D-D is typically approximately 2.0 to 3.5 millimeters.

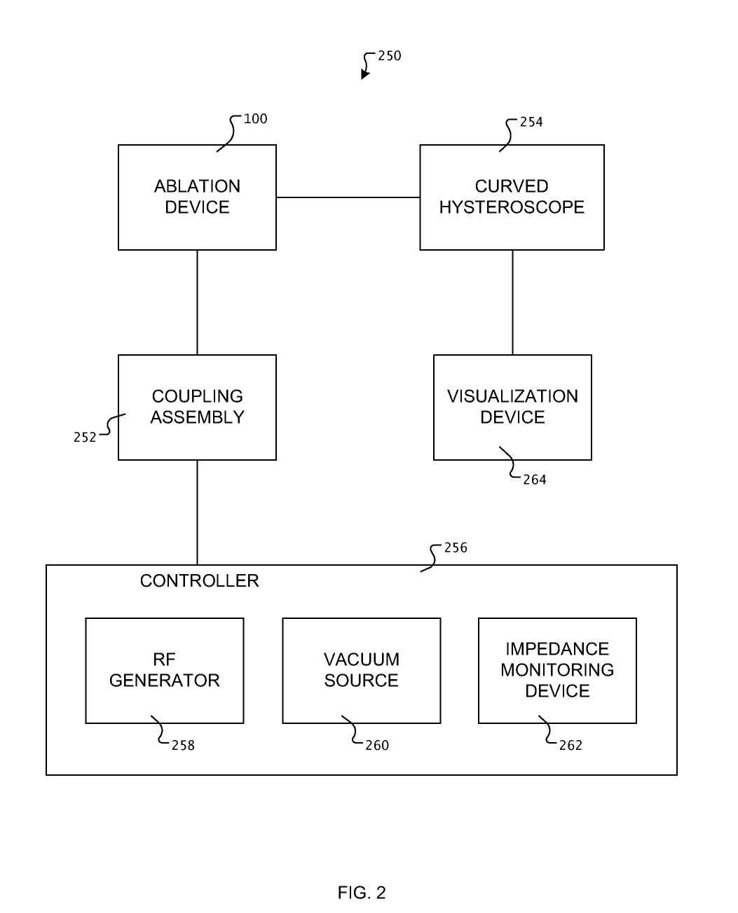

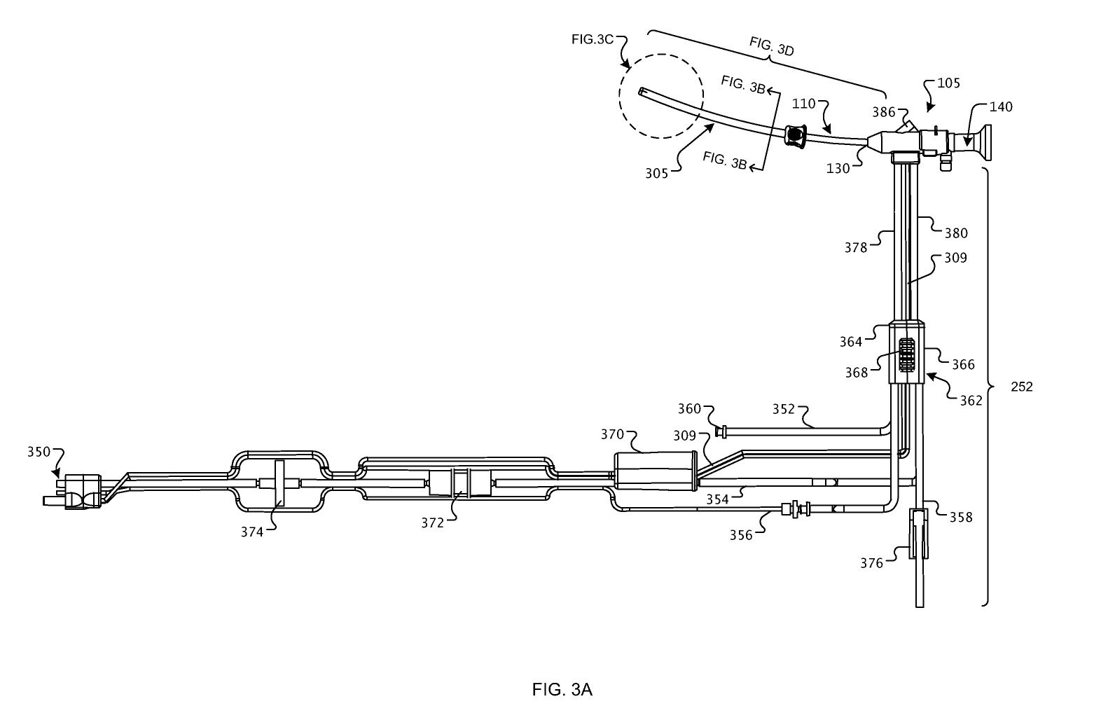

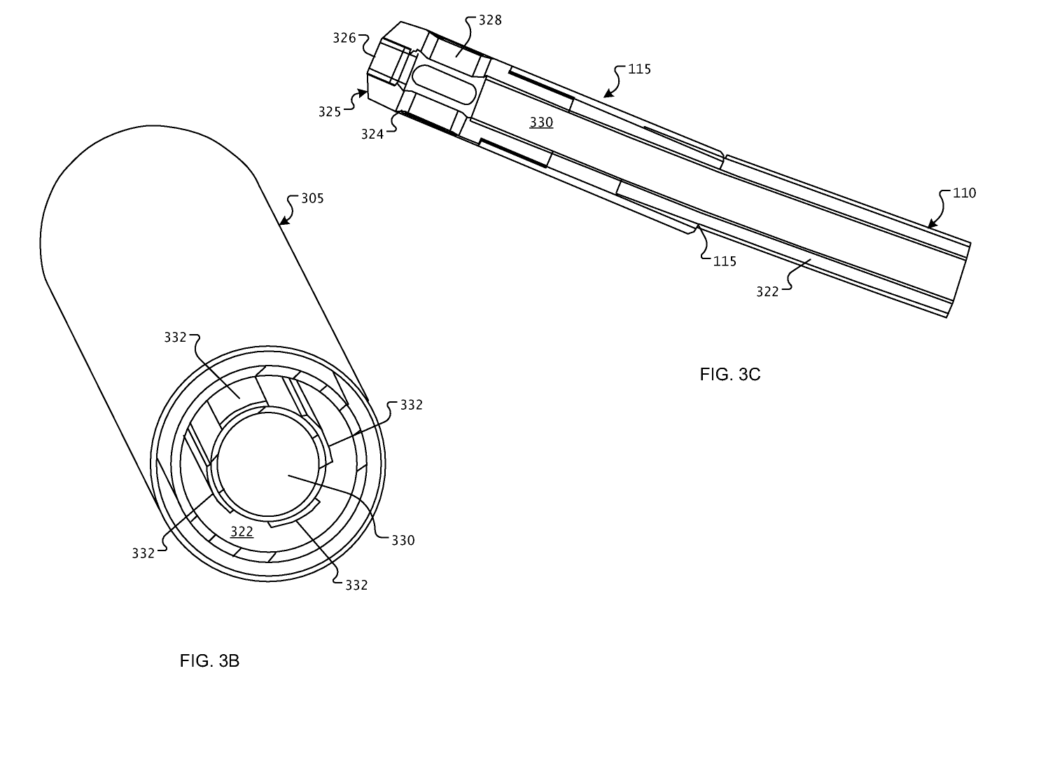

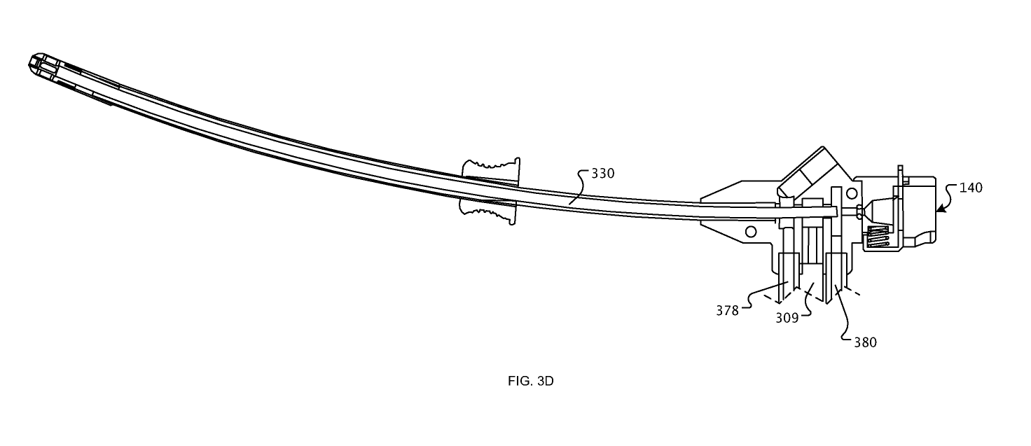

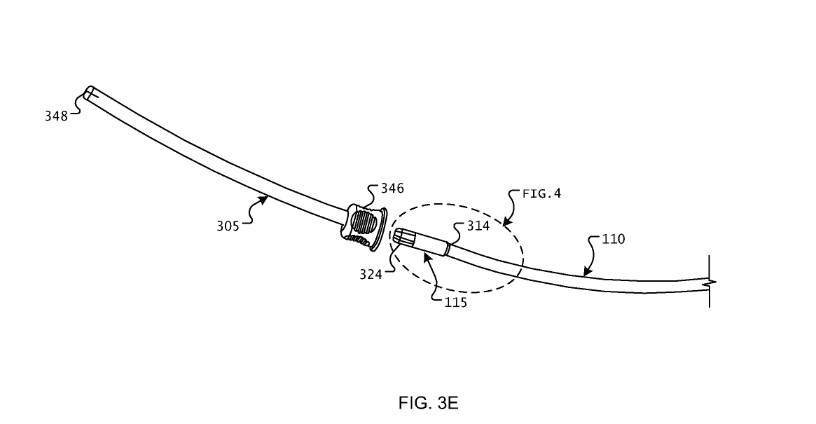

In reference to FIG. 3A, the handle 105 is configured to couple the ablation device 100 to the curved hysteroscope, which can be received via a port 140, and to a coupling assembly to couple the ablation device to a controller. Referring to FIG. 2, a schematic block diagram is shown of a system 250 for tissue ablation using the ablation device 100. The system 250 includes the ablation device 100 that is coupled to a coupling assembly 252 and configured to receive the curved hysteroscope 254. The coupling assembly 252 couples the ablation device 100 to a controller 256. The controller 256 includes an RF generator 258 and a vacuum source 260. Optionally, the controller 256 can include an impedance monitoring device 262. In one implementation, the controller 256 is a single device, however, in other implementations, the controller 256 can be formed from multiple devices coupled to one another.

Referring to FIGS. 3A-3E, one implementation of a coupling assembly 252 is shown connected to the ablation device 100 shown in FIG. 1. Other configurations of the coupling assembly 252 are possible, and the one described herein is just one example for illustrative purposes. The coupling assembly 252 as well as certain aspects of the ablation device 100 shall be described in further detail below in reference to FIGS. 3A-E.

Referring particularly to FIGS. 3B-D, a cross-sectional side view of the ablation device 100 is shown (FIG. 3D), as well as the distal ends of connectors of the coupling assembly 252. In particular, in this implementation, there are at least three connections made to the coupling assembly 252. A first connection connects the ablation device 100 to a vacuum feedback/saline supply line 378. A second connection connects the ablation device 100 to an RF cable bundle 309. A third connection connects the ablation device 100 to a suction/waste line 380.

The vacuum feedback/saline supply line 378 fluidly couples to an outer lumen 322 formed in the curved shaft 110, shown in the cutaway view in FIG. 3B. As described further below, saline can be supplied to the distal end of the ablation device 100 and into the uterine cavity to distend the cavity during a medical procedure. The RF cable bundle 309 is electrically connected to connectors 332 that run from the RF applicator head 115 to the proximal end of the ablation device 100, and provides RF power to the one or more bipolar electrodes, as described further below. The suction/waste line 380 is fluidly coupled to an inner lumen 330 included in the curved shaft 110, and provides suction to the RF applicator head to maintain the one or more bipolar electrodes in contact with surrounding tissue as well as removing liquid and liberated steam during an ablation procedure. The connectors 332 can be conductive elements formed on the outer surface of an insulating tube that provides the inner lumen 330. The proximal end of the ablation device 100 includes a port 140 configured to receive the hysteroscope 254 into the inner lumen 330 of the ablation device 100.

Referring to FIG. 3C, a cross-sectional side view of the RF applicator head 115 is shown. The inner lumen 330 in the curved shaft 110 extends through the RF applicator head 115 to the distal tip 326. When the hysteroscope 254 is positioned within the inner lumen 330, a distal end of the hysteroscope 254 sits just proud the distal tip 326 of the ablation device 100, providing for visualization from the distal tip 326 of the device 100.

Referring to FIG. 3E, a protective sheath 305 facilitates insertion of the ablation device 100 into, and removal of the ablation device 100 from, the uterine cavity 225. The protective sheath 305 is a tubular member that is slidable over the curved shaft 110 and includes a collar 346 and an expandable tip 348. The protective sheath 305 is slidable between a distal condition, shown in FIG. 3A, in which the RF applicator head 115 is inside the sheath, and a proximal condition in which the protective sheath 305 is moved toward the proximal end of the curved shaft 110. The expandable tip 348 opens so as to release the RF applicator head 115 from inside the protective sheath 305. By inserting the RF applicator head 115 into protective sheath 305, the RF applicator head 115 can be easily inserted transcervically into the uterine cavity 225.

During use, the protective sheath 305 is retracted from the RF applicator head 115, for example, by grasping the collar 346 and moving the protective sheath 305 toward the proximal end of the curved shaft 110. Alternatively, moving the handle 105 toward the collar 346 can also advance the curved shaft 110 relative to the sheath 305, thereby exposing the RF applicator head 115.

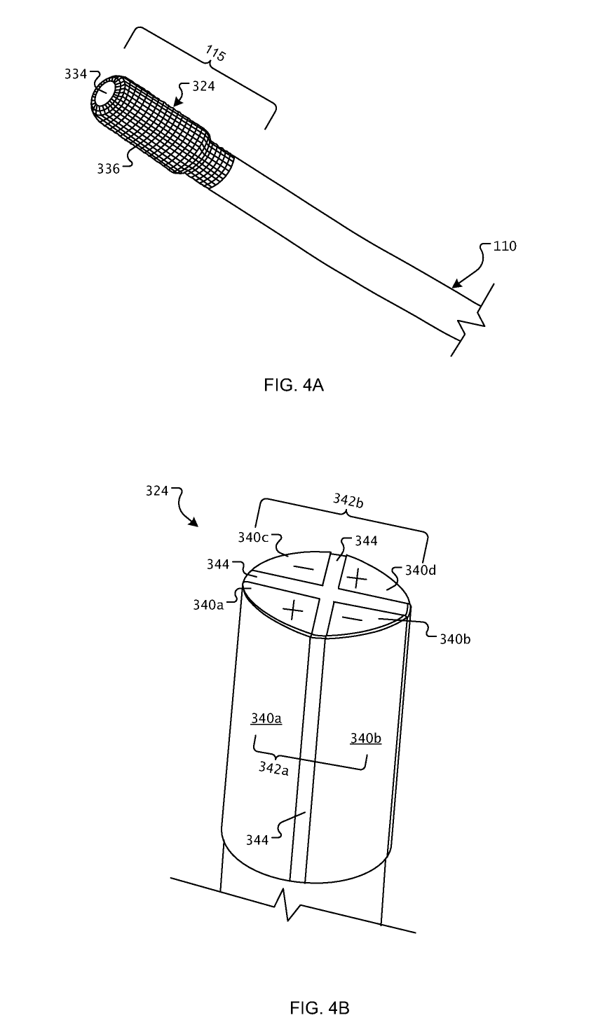

Referring to FIG. 4A, a close up view of the RF applicator head 115 is shown including an electrode carrier 324. FIG. 4B shows a schematic representation of the electrode carrier 324 including conductive regions forming bipolar electrodes 342a and 342b and non-conductive regions 344 providing insulation therebetween. In the current embodiment, the electrode carrier 324 includes an approximately cylindrically shaped support member within a fabric sheath 336. The fabric sheath 336 includes conductive metallized regions 340a-d separated by a non-conductive region 344 formed onto the fabric sheath 336. A pair of electrodes, i.e., one positively charged and the other negatively charged, together form one bipolar electrode. In the embodiment shown, the electrode pair 340a and 340b together form a bipolar electrode 342a, and the electrode pair 340c and 340d together from a bipolar electrode 342b. In one implementation, the electrode carrier 324 has a diameter in the range of approximately five to ten millimeters, for example, six millimeters. However, it should be noted that other sizes and configurations are possible. For example, the electrode carrier can be an approximately tapered cylindrical support member within a fabric sheath.

In another implementation, the electrode carrier 324 can be formed from a metallic mesh insert molded into a support member formed from a plastic material. The metallic mesh insert forms the electrically conductive regions (i.e., electrodes 340a-d) and the plastic material forms the non-conductive regions (i.e., insulator 344) thereby creating the one or more bipolar electrodes (i.e., bi-polar electrodes 342a and 342b). The metallic mesh insert can be formed from an electrically conductive material such as a stainless steel material, a platinum material, or other electrically conductive materials.

Referring again to the embodiment of the electrode carrier 324 formed from a fabric sheath 336 stretched over a support member, in one implementation, the fabric sheath 336 is formed from a nylon mesh, and the conductive metallized regions are formed by coating the nylon mesh with gold. In one embodiment, the fabric sheath 336 is formed from a composite yarn with a thermoplastic elastomer (TPE) core and multiple polyfilament nylon bundles wound around the TPE as a cover. The nylon bundles are plated with thin conductive metal layers. Preferably, the nylon is metallized, but not the TPE core. In another embodiment, nylon filaments are coated with a silver and/or gold coating. The filaments are sewn or knitted together with a non-conductive nylon or spandex filament to form the bipolar fabric sheath.

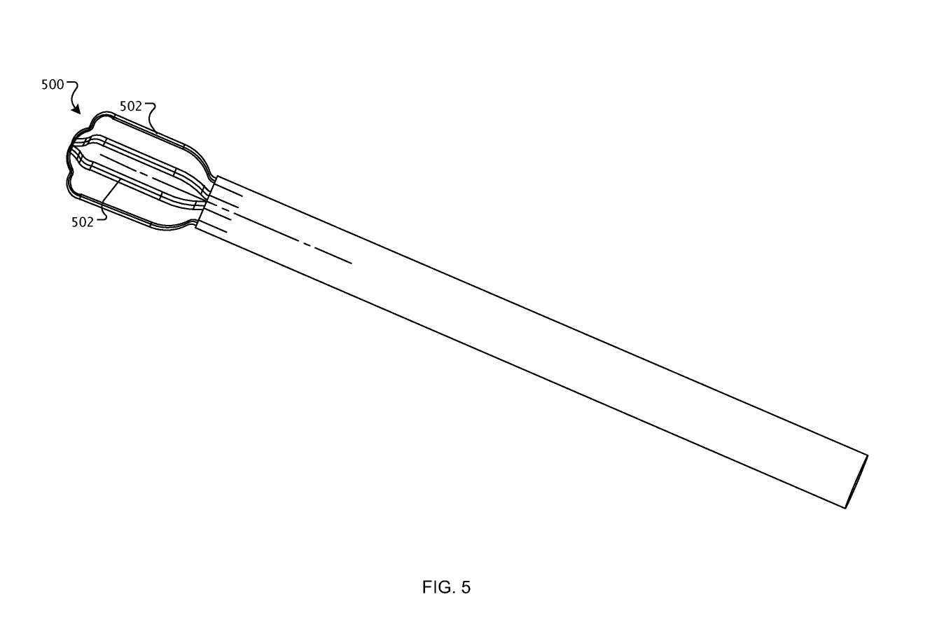

In another embodiment, the electrode carrier can be placed over an expandable or self-expandable support member. Referring to FIG. 5, the support member 500 can have a series of expandable arms 502 that when housed in an outer sheath are in a collapsed state. Once the device is inserted into the uterine cavity, the outer sheath can be withdrawn to expose the electrode array and allow the support member arms to expand. This can be advantageous to have a smaller diameter insertion profile and allow increased electrode spacing, thereby generating a deeper ablation profile. In one implementation, the support member can be fabricated from Nitinol, Elgiloy or another shape memory alloy.

The support member included in the electrode carrier 324 can be formed from any suitable material, one example being Ultem®, a thermoplastic PolyEtherImide (PEI) that combines high strength and rigidity at elevated temperatures with long term heat resistance (Ultem is a registered trademark of General Electric Company Corporation of New York, N.Y.).

In an alternative embodiment, the electrode carrier 324 can be a sack formed of a material that is non-conductive, and that is permeable to moisture. Examples of materials for the electrode carrier 324 include foam, cotton, fabric, or cotton-like material, or any other material having the desired characteristics. The electrodes 340a-d can be attached to the outer surface of the electrode carrier 324, e.g., by deposition or another attachment mechanism. The electrodes 340a-d can be made of lengths of silver, gold, platinum, or any other conductive material. The electrodes 340a-d can be formed on the electrode carrier 324 by electron beam deposition, or they can be formed into coiled wires and bonded to the electrode carrier 324 using a flexible adhesive. Other means of attaching the electrodes 340a-d, such as sewing them onto the surface of the electrode carrier 324, may alternatively be used.

The depth of destruction of the target tissue can be controlled to achieve repeatable, predetermined depths. Variables such as the electrode construction, power applied to the electrodes 340a-d (power density or power per unit surface area of the electrode), and the tissue impedance at which power is terminated can be used to affect the depth of tissue destruction, as discussed further below.

Still referring to FIG. 4B, the spacing between the electrodes 340a-d (i.e., the distance between the centers of adjacent electrodes) and the widths of the electrodes 340a-d are selected so that ablation will reach predetermined depths within the tissue, particularly when maximum power is delivered through the electrodes 340a-d. Maximum power is the level at which low impedance, low voltage ablation can be achieved. The depth of ablation is also affected by the electrode density (i.e., the percentage of the target tissue area which is in contact with active electrode surfaces) and may be regulated by pre-selecting the amount of active electrode coverage. For example, the depth of ablation is much greater when the active electrode surface covers more than 10% of the target tissue than it is when the active electrode surfaces covers only 1% of the target tissue.

By way of illustration, using 3-6 mm spacing, an electrode width of approximately 0.5-2.5 mm and a delivery of approximately 20-40 watts over a 9-16 cm² target tissue area, will cause ablation to a depth of approximately 5-7 millimeters when the active electrode surface covers more than 10% of the target tissue area. After reaching this ablation depth, the impedance of the tissue will become so great that ablation will self-terminate. By contrast, using the same power, spacing, electrode width, and RF frequency will produce an ablation depth of only 2-3 mm when the active electrode surfaces covers less than 1% of the target tissue area.

Referring again to FIG. 3A, the coupling assembly 252 shall be described in further detail. The RF cable bundle 309 includes one or more electrical conductors (i.e., wire, flexible circuit, stripline, or other) that electrically connect to the electrical conductors 332 included in the ablation device 100. The RF cable bundle 309 connects at the distal end 350 of the coupling assembly 252 to the controller 256, which is configured to control the delivery of radio frequency energy to the RF applicator head 115.

The coupling assembly 252 further includes a saline supply line 352 and a vacuum feedback line 356 that merge proximal to a fluid control switch 362 to form the vacuum feedback/saline supply line 378. The vacuum feedback/saline supply line 378 is coupled to the outer lumen 322 included in the curved shaft 110 of the ablation device 100. The controller 256 is in communication with and receives a vacuum feedback signal from the vacuum feedback line 356. The vacuum feedback line 356 allows the controller 256 to monitor the vacuum level at the ablation site. The saline supply line 352 includes a connector 360 (e.g., female luer, threaded connection, or other) located on the distal end of the saline supply line 352. The connector 360 can be removably coupled to a saline supply source (i.e., intravenous bag, or other). The fluid control switch 362 can control the flow of fluid (i.e., saline) to the ablation site and, in one embodiment, includes a roller clamp body top half 364, a roller clamp body bottom half 366, and a roller wheel 368.

The coupling assembly 252 further includes a waste line 358 and suction line 354. The suction line 354 and the waste line 358 merge proximal to the fluid control switch 362 to form the suction/waste line 380. The suction/waste line 380 is coupled to the inner lumen 330 included in the curved shaft 110 of the ablation device 100.

The suction/waste line 380 couples to a vacuum source 260 (FIG. 2). The vacuum source 260 can be operated by the controller 256 to draw the tissue surrounding the electrode carrier 324 into contact with the one or more bipolar electrodes 342a-b. Additionally, the vacuum source 260 can draw the moisture that can be generated during the delivery of the radio frequency energy to the one or more bipolar electrodes 342a-b away from the one or more bipolar electrodes 342a-b. Further, the vacuum source 260 can substantially eliminate the liquid surrounding the one or more bipolar electrodes 342a-b. The moisture is drawn by the vacuum source 260 through the inner lumen 330, to the suction/waste line 380 and removed via the waste line 358. The waste line 358 can include a waste line roller clamp 376 that can be used to control the flow of waste, fluid, or both that is removed by the ablation device 300 from the tissue ablation site. The vacuum relief valve 386 included in the handle 105 of the ablation device 100 is in fluid communication with the suction/waste line 380 and can aid in relieving excess vacuum.

The suction line 354 can include a suction canister 370, a desiccant 372, and a filter 374. The suction canister 370 can operate as a reserve and be used to smooth out the level of vacuum applied to the ablation site. The desiccant 372 can serve to substantially dry out or absorb at least a portion of the moisture that can be contained in the fluid evacuated from the ablation site by the vacuum source 260. The filter 374 can serve to prevent any particulate matter evacuated from the ablation site by the vacuum source 260 from being communicated to the controller 256, the vacuum source 260, or both.

Referring again to FIG. 2, a hysteroscope 254 is configured to position within the inner lumen 330 of the curved shaft 110. In one embodiment, the hysteroscope 254 is substantially rigid and is configured with a curve that is substantially similar to the curve of the curved shaft 110. The curved hysteroscope 254 can be formed including optics similar to a conventional straight hysteroscope, that is, the scope can have a conventional lens system including an objective lens and a series of relay and filed lenses, to transfer the image to the camera focal plane. The relay and field lenses can be fabricated from glass elements in a typical fashion (e.g., ground and polished) and assembled with a series of spacers. The advantage of such a device is the high resolution. In another embodiment, the shaft 110 is not flexible and takes on the curve of the hysteroscope 254 upon positioning the hysteroscope 254 therein.

In yet another embodiment, the hysteroscope 254 is flexible and can flex to accommodate the curve of the curved shaft 110. In this configuration, the scope has an objective lens coupled to an image guide, e.g., a coherent bundle of fibers. The objective lens images the object to the distal end of the image guide. The individual fibers transfer the image to the proximal surface of the image guide. Additional optics are used to transfer the image to either the user's eye or the camera focal plane. The advantage of this type of scope is the scope's flexibility and ability to fabricate small diameter devices.

The hysteroscope 254 generally has an optical system that is typically connected to a video system and a light delivery system. The light delivery system is used to illuminate the target site under inspection. Referring again to the system 250 shown in FIG. 2, the hysteroscope 254 can be coupled to an external visualization device 264, for example, a monitor, to provide viewing by the operator. In some embodiments, the light source is outside of the patient's body and is directed to the target site under inspection by an optical fiber system. The optical system can include a lens system, a fiberscope system, or both that can be used to transmit the image of the organ to the viewer.

In one implementation, the ablation device 100 shown in FIG. 1A can have a curved shaft 110 that is approximately 30 centimeters long and a cross-sectional diameter of approximately 4 millimeters. The curved shaft 110 can be formed from Stainless Steel 300 series, Nitinol, Elgiloy or other metals and the handle 105 can be formed from plastic or metal, including Stainless Steel 300 series, ABS plastic, Ultem, polycarbonate, Styrenes or other machinable or moldable plastics. The sheath 305 can be formed from PET, TFE, PTFE, FEP, or polyolefin. Components of the coupling assembly 252 can be formed from Tygon tubing and/or PVC tubing.

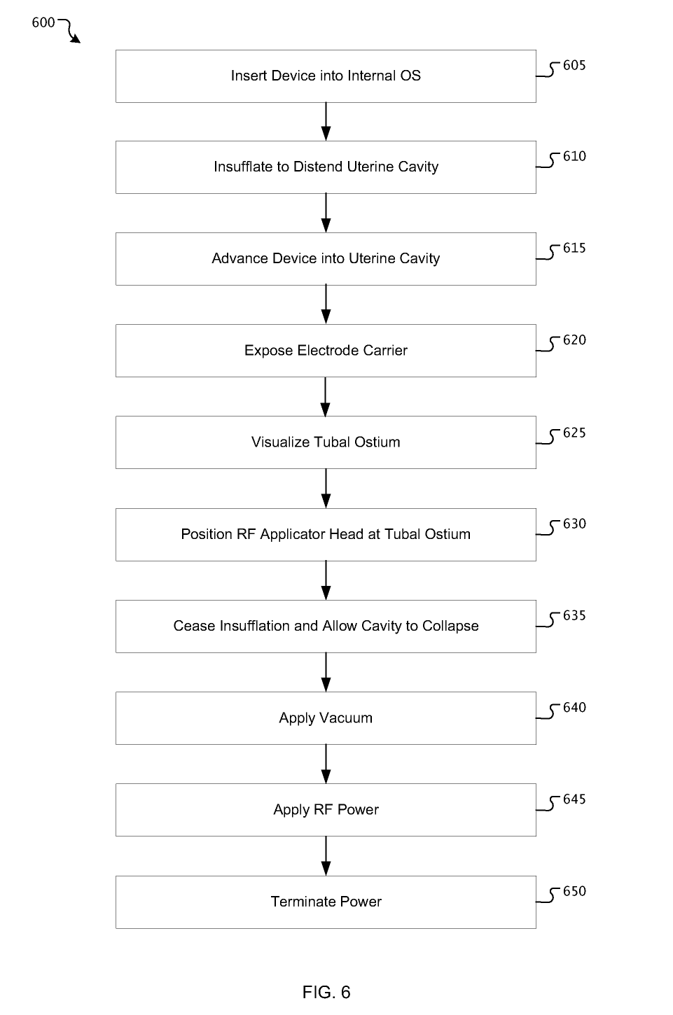

Referring to FIG. 6, an exemplary process 600 for using the ablation device 100 to sterilize a female shall be described. The distal end of the ablation device 100 is inserted through the vagina and cervix to the internal os 207 at the base of the uterus 200 (step 605). A gas, e.g., carbon dioxide, or a liquid, e.g., saline, is delivered into the uterine cavity 225 via the vacuum feedback/saline supply line 378 to distend the uterine cavity 225 (step 610). The ablation device 300 is then advanced into the uterine cavity 225 (step 615). The protective sheath 305 is withdrawn to expose the RF applicator head 115 and, in particular, the electrode carrier 324 positioned at the distal end thereof (step 620).

The hysteroscope 254, which is advanced into the inner lumen 330 of the ablation device 100, is used to visualize the target tubal ostium 230 (step 625). In the system shown in FIG. 2, the hysteroscope 254 communicates with an external visualization device 264. The operator can thereby view advancement of the distal end of the ablation device 100 toward a tubal ostium 230. The distal tip of the RF applicator head 115, which is still within the protective sheath 305, is positioned at the tubal ostium 230 (step 630).

Insufflation is ceased and the uterine cavity 225 is allowed to collapse onto the RF applicator head 115 (step 635). The fluid control switch is switched to allow for suction/aspiration and waste management. Vacuum can be applied to the RF applicator head 115 via the suction/waste line 380 to draw the surrounding tissue into contact with the electrodes 340a-d (step 640). The RF generator 258 is turned on to provide RF energy to the electrodes 340a-d (step 645). The RF energy is ceased once the desired amount of tissue has been ablated (step 650). In one implementation, 5 watts of RF power is supplied per square centimeter of electrode surface area until the predetermined impedance threshold is reached, at which point power is terminated.

In one implementation, to achieve the desired depth of ablation, the controller 256 is configured to monitor the impedance of the tissue at the distal end of the RF applicator head 115, for example, using an impedance monitoring device 262 (FIG. 2). The controller 256 can include an automatic shut-off once a threshold impedance is detected. As the tissue is desiccated by the RF energy, fluid is lost and withdrawn from the region by a vacuum through the inner lumen 330 and the suction/waste line 380. The suction draws moisture released by tissue undergoing ablation away from the electrode carrier 324 and prevents formation of a low-impedance liquid layer around the electrodes 340a-d during ablation. As more tissue is desiccated, the higher the impedance experienced at the electrodes 340a-d. By calibrating the RF generator 258, taking into account system impedance (e.g., inductance in cabling etc.), a threshold impedance level can be set that corresponds to a desired depth of ablation.

Once the threshold impedance is detected, the controller 256 shuts off the RF energy, preventing excess destruction of tissue. For example, when transmitting RF energy of 5 watts per square centimeter to tissue, an impedance of the tissue of 50 ohms can indicate a depth of destruction of approximately 3 to 4 millimeters at the proximal end and approximately 2.5 millimeters at the distal end. In an alternative embodiment, the RF generator 258 can be configured such that above the threshold impedance level the RF generator's ability to deliver RF power is greatly reduced, which in effect automatically terminates energy delivery. The uterine cavity 225 can be insufflated a second time, and the ablation device 100 rotated approximately 180° to position the RF applicator head 115 at the other tubal ostium 230 and the above procedure repeated to ablate tissue at the other tubal ostium 230. The hysteroscope 254 is reinserted to guide repositioning of the head 115 to the second tubal ostium. The ablation device 100 is then withdrawn from the patient's body. After ablation, healing and scarring responses of the tissue at the tubal ostia 230 permanently occlude the fallopian tubes 220, without requiring any foreign objects to remain in the female's body and without any incisions into the female's abdomen. The procedure is quick, minimally invasive and is highly effective at tubal occlusion.

Optionally, a constant rate of RF power can be supplied for a first time period following which the RF power can be increased, either gradually or abruptly, for a second time period. Although the system 250 includes a vacuum source to transport moisture away from the tissue site during ablation, after the first time period, the impedance at the RF applicator head may decrease due to fluid migration into the site. Increasing the RF power at this point for the second time period can help to vaporize the excess fluid and increase the impedance. The RF power can be increased as described in U.S. patent application Ser. No. ______, entitled “Power Ramping During RF Ablation”, filed ______, by Kotmel et al, the entire contents of which are hereby incorporated by reference herein.

In one embodiment, ramping up the RF power density includes steadily or gradually increasing the current over a second time period after an initial time period. Determining when to begin the power ramp-up, i.e., determining the value of the initial time period, and the amount by which to ramp-up, in one implementation is according to a time-based function and in another implementation is according to an impedance-based function.

In one implementation, the RF power density applied to the tissue ablation site is substantially constant at value PD₁ for the duration of a first time period of n seconds. At the end of the first time period, the RF power density is ramped up at a substantially constant and gradual rate to a value PD₂ for the duration of a second time period. The power ramping rate can be linear, however, in other implementations, the power can be ramped at a non-linear rate.

The duration of the first time period, i.e., n seconds, is a time after which the impedance level at the electrode/tissue interface decreases to a threshold impedance of Z₁ or by a threshold percentage level to Z₁. The value of “n” can be determined either empirically, e.g., by experimentation, or by monitoring the impedance at the electrode/tissue interface, for example, using the impedance monitoring device 262. In either case, once the threshold impedance Z₁ has been reached, the power density is ramped up to vaporize excess fluid that has likely migrated to the electrode/tissue interface and caused the decrease in impedance. The RF power density applied for the duration of the second time period is ramped up at a constant rate from PD₁ to PD₂. As fluid at the tissue ablation site is substantially vaporized by the increased power density and the tissue continues to undergo ablation, the impedance level increases. At a point in time t₂, the RF power is terminated, either based on an empirically determined time period, or based on the impedance level substantially flattening out at that point, indicating the tissue ablation process is complete.

The values of power density relative to the monitored impedance level, can be as set forth in the table below. These values are only illustrative of one implementation, and differing values can be appropriate. The depth of tissue destruction is dependent on factors other than power density, for example, electrode spacing, and thus if other factors are varied, the power density levels indicated below may change as well.

| | | Rate of Power Density |
|---|---|---|
| Initial Power Density | Drop in Impedance | Increase |
| (watts/cm²) | after first time period | ({watts/cm²}/sec) |
| 5 | 25% | 1 |
| 5 | 33% | 2–3 |

In an implementation where the values of time period and power densities are determined empirically, i.e., rather than by monitoring impedance levels, the values of time and power density in an application of tubal occlusion can be as follows. The initial RF power density can be approximately 5 watts/cm² and the initial time period “n” can be between approximately 10 and 60 seconds. After the first time period, and for the duration of the second time period, the RF power density can be increased at a rate of approximately 0.5 to 2.5 watts/cm² per second. The duration of the second time period can be between approximately 5 and 10 seconds.

In a more specific example, the initial RF power density is approximately 5 watts/cm² and the initial time period is between approximately 45 and 60 seconds. After the first time period, and for the duration of the second time period, the RF power density is increased at a rate of approximately 1 watt/cm² per second. The duration of the second time period is between approximately 5 and 10 seconds.

In another implementation, the RF power density applied to the tissue ablation site is substantially constant at PD₁ for a first time period. At time t₁, in response to a sudden and significant decrease in impedance from Z₀ to Z₁, the RF power density is abruptly ramped up to a level PD₂. The level PD₂ can be empirically determined in advance or can be a function of the percentage in decrease of the impedance level.

In one implementation, the RF power density is held at the level PD₂ until the impedance increases to the level it was at prior to the sudden and significant decrease, i.e., Z₀. The RF power density is then returned to the initial level PD₁. Optionally, the RF power density can then be gradually ramped up for another time period from PD₂ to PD₃. The gradual ramp up in RF power density can start immediately, or can start after some time has passed. Once the impedance reaches a threshold high at Z₃ (and/or flattens out), the tissue ablation is complete and the RF power is terminated.

In yet another implementation, the RF power density can be applied to the tissue ablation site at a substantially constant value (i.e., PD₁) for the duration of a first time period until a time t₁. At time t₁, in response to the impedance level being detected as suddenly and significantly decreasing from Z₀ to Z₁, the RF power density is abruptly ramped up to a level PD₂. In this implementation, the RF power density is maintained at the level PD₂ until the impedance reaches a threshold high and/or flattens out at Z₂. At this point, the tissue ablation is complete and the delivery of RF power is terminated.

By way of illustration, in one implementation, the initial power density PD₁ is approximately 5 watts/cm². Upon detecting a decrease in the impedance level by approximately 50% or more, the power density is ramped up to PD₂ which is in the range of approximately 10-15 watts/cm². After the impedance level has returned to approximately the initial pre-drop level of Z₀, the power density is returned to PD₁ of approximately 5 watts/cm². Optionally, the power density can then be ramped up, either immediately or after a duration of time, at a rate of approximately 1 watt/cm² per second. These values are only illustrative of one implementation, and differing values can be appropriate. The depth of tissue destruction is dependent on factors other than power density, for example, electrode spacing, and thus if other factors are varied, the power density levels indicated below may change as well.

As discussed above, in an alternative embodiment the curved endoscopic device can be configured as a curved endoscope that includes a working channel to receive a tool for performing a medical procedure. For illustrative purposes, referring to the ablation device 100, an alternative configuration would include a curved hysteroscope with a working channel configured to receive an ablation device similar to the ablation device 100, i.e., the reverse of the ablation device 100, which includes an inner lumen 330 to receive a hysteroscope. In other implementations, the curved endoscopic device can be configured as a curved endoscope adapted to be received by a body cavity other than a uterus, for example, by a nasal passage. The working channel can be adapted to receive a tool other than an ablation device, depending on the medical procedure to be performed within the nasal passage.

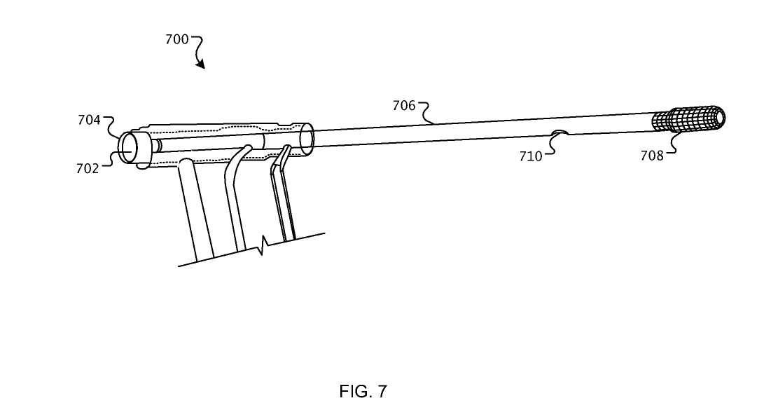

Referring to FIG. 7, an alternative embodiment of an ablation device 700 is shown. The ablation device 700 includes a port 702 configured to receive an endoscope and a mating connector 704 configured to mate with and connect to the endoscope. The port 702 is connected to a lumen formed within a shaft 706. An electrode carrier 708 is positioned at the distal end of the shaft 706. The shaft 706 of the ablation device 700 includes a side hole 710 that is proximal to the electrode carrier 708. An endoscope can be inserted into the port 702 and advanced along the length of the inner lumen toward the side hole 710 formed in the shaft 706. The distal end of the endoscope can be passed through the side hole 710 to provide the endoscope with an orientation whereby the distal end of the endoscope is substantially parallel to the shaft 706 of the ablation device 700. The shaft 706 is flexible, and can be formed from a polymer. The action of inserting a rigid endoscope into the lumen formed in the shaft 706 curves the shaft 706 at its distal end, deflecting the distal tip of the ablation device in a direction opposite the endoscope position. That is, the shaft 706 can be flexible but elastic with restorative forces to urge the shaft 706 to a shape that is substantially straight.

The distal end of the endoscope includes optics (e.g., lens, fiber optics, or other) to provide visualization when positioning the electrode carrier 708 at an ablation side. The side-by-side configuration of the endoscope optics and the electrode carrier 708 can provide the user with off-axis viewing. For example, the endoscope can have off-axis viewing in the range of ten degrees to ninety degrees, and such off-axis viewing can help the user to align the electrode carrier 708 with an ablation sight, for example, the tubal ostium of a fallopian tube.

The ablation device 700 can be configured to mate with a coupling assembly similar to the coupling assembly described in reference to FIG. 3A, or a differently configured coupling assembly, which couples the ablation device 700 to a controller including or connected to an RF generator, vacuum source and optionally an impedance monitoring device. In another embodiment, the ablation device 700 can be configured with a curve, for example, in one implementation a curve to facilitate insertion into a uterine cavity or another body cavity.

A number of embodiments of the invention have been described. Nevertheless, it will be understood that various modifications may be made without departing from the spirit and scope of the invention. Accordingly, other embodiments are within the scope of the following claims.

What is claimed is:

1. An apparatus for occluding a fallopian tube, comprising:
an elongate member having a distal end, a proximal end and a central interior including at least a first lumen operable to couple to a vacuum source and to draw moisture way from one or more electrodes included in an electrode carrier positioned at the distal end of the elongate member and at least a second lumen configured to receive a hysteroscope, where the first lumen and the second lumen can be the same lumen or can be separate lumens;
an electrode carrier attached to the distal end of the elongate member and including one or more bipolar electrodes formed thereon and operable to couple to a radio frequency energy generator; and
one or more conductors extending from the electrode carrier to the proximal end of the elongate member and configured to connect to a controller operable to control the delivery of radio frequency energy to the one or more bipolar electrodes;
where the elongate member is a substantially rigid member configured with a curve to facilitate advancement of the distal end transcervically through a uterus and into a region of a tubal ostium of a fallopian tube to be occluded.

2. The apparatus of claim 1, further comprising:
a hysteroscope positioned within the first lumen of the elongate member, such that a distal end of the hysteroscope is positioned approximately just proud of a distal end of the electrode carrier.

3. The apparatus of claim 2, wherein the hysteroscope is substantially rigid and configured with a similar curve to the curve of the elongate member.

4. The apparatus of claim 2, wherein the hysteroscope is substantially flexible and can flex to accommodate the curve of the elongate member.

5. The apparatus of claim 1, where the electrode carrier comprises an approximately cylindrically shaped support member within a fabric sheath having conductive metallized regions and one or more non-conductive regions formed thereon to create the one or more bipolar electrodes.

6. The apparatus of claim 5, where the support member is formed from a plastic material, the fabric sheath is formed from a polymer mesh and the conductive metallized regions are formed by selectively coating the polymer mesh with gold.

7. The apparatus of claim 6, where the polymer comprises a combination of nylon and spandex.

8. The apparatus of claim 1, where the electrode carrier is an approximately cylindrically shaped member comprising a metallic mesh insert molded in a support member formed from a plastic material and where the metallic mesh forms conductive regions and the plastic material forms non-conductive regions thereby creating the one or more bipolar electrodes.

9. The apparatus of claim 8, where the metallic mesh insert is formed from a stainless steel material.

10. The apparatus of claim 8, where the metallic mesh insert is formed from a platinum material.

11. The apparatus of claim 1, where the electrode carrier comprises an approximately cylindrically shaped support member having a diameter in the range of approximately five to 10 millimeters.

12. The apparatus of claim 1, further comprising:
a vacuum source in fluid communication with the first lumen included in the elongate member and operable to draw tissue surrounding the electrode carrier into contact with the one or more bipolar electrodes and to draw moisture generated during delivery of the radio frequency energy to the one or more bipolar electrodes away from the one or more bipolar electrodes and to substantially eliminate liquid surrounding the one or more bipolar electrodes.

13. The apparatus of claim 1, further comprising:
a radio frequency energy generator coupled to the one or more bipolar electrodes through the one or more conductors, where the radio frequency energy generator includes or is coupled to a controller operable to control the delivery of radio frequency energy to the one or more bipolar electrodes.

14. An apparatus for occluding a fallopian tube, comprising:
a hysteroscope including a working channel extending from a distal end to a proximal end, where the hysteroscope is substantially rigid and configured with a curve to facilitate advancement of the distal end transcervically through a uterine cavity and into a region of a tubal ostium of a fallopian tube to be occluded;
an elongate member positioned within the working channel of the hysteroscope, the elongate member having a distal end, a proximal end and a central interior including a lumen operable to couple to a vacuum source and to draw moisture way from one or more electrodes included in an electrode carrier positioned at the distal end of the elongate member and where the elongate member is a substantially rigid member configured with a curve similar to the curve of the hysteroscope to facilitate advancement of the distal end of the elongate member to the distal end of the hysteroscope;
an electrode carrier attached to the distal end of the elongate member and including one or more bipolar electrodes formed thereon and operable to couple to a radio frequency energy generator; and
one or more conductors extending from the electrode carrier to the proximal end of the elongate member and configured to connect to a controller operable to control the delivery of radio frequency energy to the one or more bipolar electrodes.

15. An apparatus for ablating tissue, comprising:
an elongate member having a distal end, a proximal end and a central interior including at least a first lumen operable to couple to a vacuum source and to draw moisture way from one or more electrodes included in an electrode carrier positioned at the distal end of the elongate member and at least a second lumen configured to receive an endoscope;
an electrode carrier attached to the distal end of the elongate member and including one or more bipolar electrodes formed thereon and operable to couple to a radio frequency energy generator; and
one or more conductors extending from the electrode carrier to the proximal end of the elongate member and configured to connect to a controller operable to control the delivery of radio frequency energy to the one or more bipolar electrodes;
where the elongate member is a substantially rigid member configured with a curve to facilitate advancement of the distal end through a body cavity to a region of tissue to be ablated.

16. An apparatus for ablating tissue, comprising:
an endoscope including a working channel extending from a distal end to a proximal end, where the endoscope is substantially rigid and configured with a curve to facilitate advancement of the distal end through a body cavity to a region of tissue to be ablated;
an elongate member positioned within the working channel of the endoscope, the elongate member having a distal end, a proximal end and a central interior including a lumen operable to couple to a vacuum source and to draw moisture way from one or more electrodes included in an electrode carrier positioned at the distal end of the elongate member and where the elongate member is a substantially rigid member configured with a curve similar to the curve of the hysteroscope to facilitate advancement of the distal end of the elongate member to the distal end of the endoscope;
an electrode carrier attached to the distal end of the elongate member and including one or more bipolar electrodes formed thereon and operable to couple to a radio frequency energy generator; and
one or more conductors extending from the electrode carrier to the proximal end of the elongate member and configured to connect to a controller operable to control the delivery of radio frequency energy to the one or more bipolar electrodes.

17. An apparatus for occluding a fallopian tube, comprising:
an elongate member having a distal end, a proximal end and a central interior including at least a first lumen operable to couple to a vacuum source and to draw moisture way from one or more electrodes included in an electrode carrier positioned at the distal end of the elongate member and at least a second lumen configured to receive a hysteroscope, where the first lumen and the second lumen can be the same lumen or can be separate lumens;
an electrode carrier attached to the distal end of the elongate member and including one or more bipolar electrodes formed thereon and operable to couple to a radio frequency energy generator, where the electrode carrier has a substantially cylindrical shape; and
one or more conductors extending from the electrode carrier to the proximal end of the elongate member and configured to connect to a controller operable to control the delivery of radio frequency energy to the one or more bipolar electrodes;
where the elongate member includes an aperture formed in a sidewall of the elongate member toward a distal end of the elongate member but proximate to the electrode carrier, the aperture configured to allow a distal end of the hysteroscope to pass through, providing the hysteroscope with a field of view extending from a side of the elongate member.

18. The apparatus of claim 17, where the elongate member is flexible and receiving the hysteroscope in the second lumen causes the elongate member to bend off axis forming a curvature in the elongate member.

19. An apparatus for occluding a fallopian tube, comprising:
an elongate member having a distal end, a proximal end and a central interior including at least a first lumen operable to couple to a vacuum source and to draw moisture way from one or more electrodes included in an electrode carrier positioned at the distal end of the elongate member and at least a second lumen configured to receive a rigid and curved hysteroscope, where the first lumen and the second lumen can be the same lumen or can be separate lumens;
an electrode carrier attached to the distal end of the elongate member and including one or more bipolar electrodes formed thereon and operable to couple to a radio frequency energy generator; and
one or more conductors extending from the electrode carrier to the proximal end of the elongate member and configured to connect to a controller operable to control the delivery of radio frequency energy to the one or more bipolar electrodes;
where the elongate member is a substantially flexible member configured to bend into a curved configuration upon receiving the rigid and curved hysteroscope in the second lumen, where the curve facilitates advancement of the distal end transcervically through a uterus and into a region of a tubal ostium of a fallopian tube to be occluded.

20. A method for fallopian tubal occlusion, comprising:
inserting a substantially rigid, curved elongate member including a substantially cylindrically shaped electrode carrier positioned at a distal end with one or more bipolar electrodes formed thereon into a uterine cavity;
positioning the electrode carrier at a tubal ostium of a fallopian tube such that a distal end of the electrode carrier advances into the tubal ostium; and
passing radio frequency energy through the one or more bipolar electrodes to the tubal ostium to destroy tissue to a known depth and to precipitate a healing response in surrounding tissue that over time scars and occludes the fallopian tube.

21. The method of claim 20, wherein passing radio frequency energy through the one or more bipolar electrodes comprises:
passing a current at an initial current level through the one or more bipolar electrodes to the target tissue site to apply an initial power density to destroy tissue for an initial time period; and
after the initial time period, ramping up the power density by increasing the current passed through the one or more bipolar electrodes to the target tissue site for a second time period.

22. The method of claim 21, wherein ramping up the power density comprises gradually increasing the current over the second time period.

23. The method of claim 21, wherein ramping up the power density comprises suddenly increasing the current from the initial current level to a second current level and applying the second current level for the second time period.

24. The method of claim 21, further comprising:
monitoring an impedance level at an interface between the electrode carrier and the tubal ostium;
where the initial time period is a time period after which a threshold decrease in the impedance level from an initial impedance level is detected.

25. The method of claim 21, where the initial time period is determined empirically as a time period after which an initial depth of tissue destruction has been achieved.
